(19) United States
(12) Patent Application Publication
(10) Pub. No.: US 2007/0021685 A1
(43) Pub. Date: Jan. 25, 2007

(54) Guidewire apparatus with an expandable portion and methods of use

(75) Inventors: Randolf Von Oepen, Los Altos Hills, CA (US); Travis Yribarren, San Mateo, CA (US); Thomas Rieth, Hirrlingen (DE); Lorcan Coffey, Tubingen (DE); Arik Zucker, Zurich (CH)

Correspondence Address: BEYER WEAVER & THOMAS LLP, P.O. BOX 70250, OAKLAND, CA, 94612-0250, US

(73) Assignee: Abbott Laboratories Abbott Vascular Devices, Redwood City, CA (US)

(21) Appl. No.: 11/429,424

(22) Filed: May 4, 2006

Related U.S. Application Data
(60) Provisional application No. 60/677,950, filed on May 4, 2005.
(60) Provisional application No. 60/736,638, filed on Nov. 15, 2005.

(51) Int. Cl. A61M 25/00

(52) U.S. Cl. 600/585

(57) ABSTRACT

A guidewire apparatus for a medical device that includes an expandable guidewire section that can be locked in an expanded state. In one use, the expandable section can be temporarily locked to a working element of a medical device to facilitate the transmission of torque from the guidewire to the medical device. Hence, this configuration may be utilized to aid in orientation of the working element with a vessel side branch by providing the user with the ability to apply a rotational force to the distal end of the stent delivery system. Another use includes docking the expandable section near an opening into a guidewire lumen at the catheter tip. This configuration is particularly useful in forming a smooth transition between the guidewire and the catheter tip to reduce the phenomenon of fish-mouthing.

### RELATED APPLICATION DATA

The present application claims priority under 35 U.S.C. §119 to U.S. Provisional Application Ser. No. 60/677,950, naming Von Oepen as the inventor, filed May 4, 2005, and entitled EXPANDABLE GUIDEWIRE, and U.S. Provisional Application Ser. No. 60/736,638, naming Von Oepen and Yribarren as the inventors, filed Nov. 15, 2005, and entitled GUIDEWIRE HAVING AN EXPANDING PORTION AND METHODS OF USE, both of which are incorporated herein by reference in their entirety and for all purposes.

### FIELD OF THE INVENTION

The invention generally pertains to guidewires, and more particularly to guidewires having an expandable distal section that can be secured in an expanded condition to facilitate the transmission of torque or axial loads to a medical device through which the guidewire is disposed, and/or reduce entanglement of the medical device with a deployed endoprosthesis device through which it is to pass.

### BACKGROUND OF THE INVENTION

A wide variety of guidewires have been developed for various medical applications. Intravascular guidewires, for example, are commonly used in conjunction with intravascular devices such as catheters to facilitate navigation through the vasculature of a patient. Because the vasculature of a patient may be very tortuous, it is desirable to combine a number of performance features in a guidewire. For example, it is sometimes desirable that the guidewire have a relatively high level of pushability and torqueability, particularly near its proximal end. It is also sometimes desirable that a device be relatively flexible, particularly near its distal end. A number of different guidewire structures and assemblies are known, each having certain advantages and disadvantages. However, there is an ongoing need to provide alternative guidewire structures and assemblies.

As described above, guidewires generally have good torque transmitting characteristics although torque applied to the guidewire is generally not transmitted to the medical device through which the wire is disposed. Therefore there is a need for a guidewire design that enables torque transmission through the guidewire and into the medical device through which the guidewire is disposed.

As mentioned, guidewires usually have a relatively high level of pushability compared to the catheters that they are used with, although axial loads applied guidewires are not normally transmitted to catheters. There is therefore a need for a guidewire design that enables axial load transmission through the guidewire and into the medical device through which the guidewire is disposed.

Another problem associated with conventional guidewires occurs when multiple guidewires are required for a medical procedure. For example, during treatment of a diseased vessel bifurcation, it is common to place a stent in a main branch, and then require access to a side branch through the placed stent cell. Typically, a first guidewire is placed within the main branch of the artery/vessel and a second guidewire is placed within the side branch. Depending on the stenting procedure applied, it may be necessary to remove or not place the second guidewire into the side branch vessel until the main branch has been stented. If a conventional stent is applied in the main branch, such as that shown and described in U.S. patent application Ser. No. 10/332,976 filed on Nov. 19, 2004 entitled “Endoprosthesis Having Foot Extensions”, the entirety of which is hereby incorporated by reference, the stent often bridges the bifurcation ostium. At the bridge location, it is not uncommon for a stent strut to at least partially block the ostium. Since the side branch guidewire (i.e., the second guidewire) must pass through the stent cell formed by this strut, it is also common in these situations, to have the guidewire in contact or in close proximity to the ostium-blocking strut.

A well-known and adopted procedure to address this problem is to utilize a Percutaneous Transluminal Coronary Angioplasty (PTCA) balloon catheter device to expand the struts and restored access to the side branch. In order to expand the struts, a second guidewire is advanced through the struts and into the side branch vessel. The balloon catheter device is advanced over the second guidewire and partially through the struts where it is expanded to deform the struts, and provide access to the side branch.

Occasionally, a tip portion of the catheter device becomes entangled with the stent struts when attempting to access the side branch. Typically, some clearance is allowed between the inner diameter of a guidewire lumen of the catheter device and the diameter of the guidewire in order to minimize friction and unobstructed movement between the catheter device and the guidewire. This clearance, however, can cause a phenomenon known as “fish-mouthing”. This occurs when the catheter tip tracks along the guidewire within a curve. Due to the clearance between the wire and catheter tip, the tip tends to flare out disproportionately on one side. As shown in FIGS. 1A and 1B, a significant clearance or gap 14 is thus formed between the guidewire 15 and the guidewire lumen 30 at the tip of the catheter device 26. For many procedures, this clearance 14 does not present any problems and most surgical procedures occur without any issues. In some cases, however, this flaring can cause difficulties when tracking the catheter device 26 past a difficult obstacle as it can catch on protruding surfaces (e.g. the struts 16 of an expanded stent 17).

In the case of a bifurcation procedure, the fish mouth effect can reduce the ability of the catheter device 26 to cross through a stent 16 into a side branch 18 of a bifurcated vessel 19. The flaring of the catheter tip can catch or snag on the stent struts 16 and impede the advancement of the catheter device 26. If the catheter device 26 cannot be passed into the vessel side branch 18, then the struts 16 of the expanded stent 17 cannot be moved via an expanded balloon disposed on the catheter device. The side branch 18 of the vessel 19, thus, may remain blocked or “jailed off” by the struts 16 of the expanded stent 17. Moreover, if the catheter tip becomes tangled on the struts this could lead to a dangerous condition of deforming and possibly dislodging the stent from the main vessel. Therefore, there is also a need for a device such as the present invention to allow the safe passage of a catheter device through stent struts into a vessel.

### SUMMARY OF THE INVENTION

The invention provides several alternative designs, materials and combinations in a guidewire with improved characteristics. One embodiment includes a guidewire apparatus configured to temporarily mate with a medical device at a distal section thereof. The medical device distal section includes a working element and an interior wall defining a guidewire lumen extending through the working element and terminating at an opening thereof. The guidewire apparatus is generally constructed in three distinct segments, a main section, an expandable section and a flexible distal tip section. The main section is constructed in accordance with known construction methods and may be fabricated of a solid member or a plurality of members, wherein an actuating lumen extends through the entire length of the main section. The expandable section is coupled to the distal portion of the main section, and operably communicates with the actuating lumen to facilitate actuation of the expandable section between an unexpanded state and an expanded state. In the expanded state, the expandable section is sized and dimensioned to temporarily mate against the interior wall of the guidewire lumen.

The expandable guidewire apparatus 20 of the present invention may be utilized in place of any conventional guidewire. It is contemplated that the guidewire of the present invention may be particularly useful in aligning and positioning a stent within a body duct. In particular it is contemplated that the present invention may be utilized in combination with a dedicated bifurcation stent system, wherein the bifurcation stent system includes an opening or a portion to be aligned with the side branch vessel. The present invention may be utilized to aid in orientation of the opening or portion with the side branch vessel by providing the user with the ability to apply a rotational force to the distal end of the stent delivery system.

Alternatively, it is contemplated that the guidewire of the present invention may be useful for transmitting axial loads to a medical device. In particular it is contemplated that the present invention may be utilized in combination with a stent system. The present invention may be utilized to aid in crossing a difficult to cross lesion with the stent system, such as a chronic total occlusion. The present invention provides the user with the ability to increase the combined system pushability thereby improving the ability of the system to cross through a resistant lesion.

In one specific embodiment, the expandable section includes a plurality of expansion elements disposed radially about a longitudinal axis of the expandable section. Thise elements are configured to expand substantially transversely with respect to the longitudinal axis from the unexpanded state and the expanded state.

Another embodiment includes an actuator member disposed between expandable section and the flexible tip section. A pull wire device is disposed in the actuating lumen of the main section, and includes a distal end joined to the actuator member.

In yet another specific configuration, the expandable section includes a tube member that defines an interior lumen substantially co-axially aligned with the main section actuating lumen. Each expansion element includes an expansion finger cantilever mounted to the tube member for movement between the unexpanded state and the expanded state. A push-wire member is slideably disposed in the actuating lumen, and includes a distal tip portion sized for sliding receipt in the interior lumen of the expandable section. The distal tip portions of the expansion fingers are angled inwardly into the interior lumen at an acute angle, such that when the push-wire it distally displaced into in contact with the respective distal tip portions of the expansion fingers, the expansion fingers are urged radially outward, in the expanded state.

Another aspect of the present invention provides a catheter system that includes a catheter device and an associated expandable guidewire device. The catheter device includes an elongated tube member, and a distal section that includes a working element associated with a distal portion of the tube member, and a catheter tip portion associated with a distal end of the working element. The catheter device further defines a guidewire lumen extending through the elongated tube member that terminates at a distal end of the catheter tip portion. The distal section includes an interior anchor wall defining a portion of the guidewire lumen. The expandable guidewire device is disposed within the guidewire lumen of the catheter device, and is configured for substantially axial displacement therein. The guidewire device includes an elongated main section having an actuating lumen extending substantially therethrough to a distal portion thereof; and an expandable section coupled to the distal portion of the elongated guidewire section. The expandable section operably communicates with the actuating lumen to facilitate selective actuation of the expandable section between an unexpanded state and an expanded state. In the expanded state, the expandable section has a transverse cross-sectional dimension greater than that in the unexpanded state and sufficient to contact and frictionally engage the interior anchor wall of the distal section by an amount such that selective torsional and/or axial forces applied to the elongated guidewire section are transmitted to the catheter tip portion of the catheter.

In one specific configuration, the interior anchor wall is composed of a material that is harder than that of the catheter tip portion to radially reinforce the catheter tip portion.

Yet another specific aspect of the present invention includes a method for temporarily and selectively mating a guidewire apparatus to a distal section of a medical device disposed in a vessel to transfer an a force from the guidewire apparatus to the medical device. The method includes positioning an expandable guidewire apparatus, having an elongated main section and an expandable section associated with the main section, in the guidewire lumen such that the expandable section is oriented proximate to the medical device distal section. The next event includes actuating the expandable section of the guidewire apparatus from an unexpanded state, sized and dimensioned for substantially interference free axial and rotational passage through the guidewire lumen, to an expanded state, sized and dimensioned to temporarily engage the guidewire lumen interior wall at the distal section of the medical device. The method further includes applying a force to the main section of the guidewire apparatus that is transmitted to the working element of the medical device via the temporarily mated expandable section of the guidewire apparatus, in the expanded state.

In one embodiment, the applying a force includes applying a torsion force to the main section. In another aspect, the applying a force includes applying an axial force to the main section.

Still another embodiment includes radially expanding a plurality of expansion elements of the expandable portion disposed radially about a longitudinal axis of the expandable section. Each expansion element is configured to elastically deform radially outward with respect to the longitudinal axis from the unexpanded state and the expanded state. In one particular embodiment, the expanding includes pulling a pull-wire proximally, having a distal end joined to an actuator member disposed distally to the expandable section, such that the actuator member substantially axially compresses the expandable section toward the expanded state. In another configuration, the expanding includes pushing a push-wire, longitudinally disposed in an actuating lumen of the guidewire main section, in a direction distally such that the push wire slideably contacts respective distal tip portions of expansion fingers of the expandable section that are angled inwardly into the interior lumen. Such contact urges the corresponding expansion finger radially outward, in the expanded state.

Another aspect of the present invention includes a method for reducing potential flaring between a guidewire and a distal section of a medical device inserted over the guidewire during a medical procedure. The distal section of the medical device includes a working element and defines a guidewire lumen extending through the working element and terminating at a distal opening thereof. The method includes orienting the guidewire, having an elongated main section and an expandable section associated with a distal portion of the main section, in the guidewire lumen such that the expandable section is positioned proximate to the distal opening into the guidewire lumen of the medical device. The method further includes actuating the expandable section of the guidewire apparatus from a base diameter unexpanded state to an expanded state. In the unexpanded state, base diameter is sized and dimensioned for substantially interference free axial and rotational passage through the guidewire lumen. In the expanded state, the expandable section is sized and dimensioned to substantially fill the guidewire lumen opening of the distal section to form a relatively smooth transition ramp. The ramp formed by the expandable section tapers radially outward, from the base diameter of the guidewire to at least substantially transverse cross-sectional dimension of the guidewire lumen opening at the distal section of the medical device.

In one specific embodiment, the method further includes applying an axial force to the medical device wherein the transition ramp formed by the expand section facilitates passage through a resistant location.

### BRIEF DESCRIPTION OF THE DRAWINGS

The assembly of the present invention has other objects and features of advantage which will be more readily apparent from the following description of the best mode of carrying out the invention and the appended claims, when taken in conjunction with the accompanying drawing, in which:

FIG. 1A is a fragmentary side elevation view, in cross-section, of an exemplary catheter device entangled in a strut of a stent while attempting to access a side branch of a bifurcated vessel.

FIG. 1B is an enlarged a side elevation view of the tip of the exemplary catheter device entangled in the stent strut, and taken along the circle of the line 1B-1B in FIG. 1A.

FIG. 2 is a side elevation view of an exemplary embodiment of an expandable guidewire apparatus, constructed in accordance with the present invention, wherein an expandable section is unexpanded.

FIG. 3 is a side elevation view of the expandable guidewire apparatus of FIG. 2, wherein the expandable section is expanded.

FIG. 4 is an enlarged, fragmentary, side elevation view, in cross-section, of the distal end of the guidewire apparatus of FIG. 2, illustrating the expandable section in an unexpanded state.

FIG. 5 is a side elevation view, in cross-section, of the distal end of the guidewire apparatus of FIG. 4, in the unexpanded state.

FIG. 6 is a partial cross-sectional view of the expanded distal end of the guidewire in accordance with the present invention, wherein the guidewire apparatus is shown disposed in a medical device.

FIG. 7 is a partial cross-sectional view of the expanded distal end of the guidewire in accordance with the present invention, wherein the guidewire apparatus is shown disposed in a medical device and a rotational force is shown being applied to the medical device by the guidewire.

FIG. 8 is an enlarged, fragmentary, side elevation view of the expanded distal end of the guidewire apparatus in accordance with the present invention, wherein the guidewire apparatus is shown disposed in a medical device to form a smooth transition than enables a stent strut to pass over without entanglement.

FIG. 9 is a fragmentary top perspective view of an alternative embodiment of FIG. 4, in the unexpanded state.

FIG. 10 is a fragmentary top perspective view of the alternative embodiment of FIG. 9, in the expanded state.

FIG. 11 is a partial plan view of an alternative embodiment of the expandable section in accordance with the present invention.

FIG. 12 is a partial plan view of another alternative embodiment of the expandable section in accordance with the present invention.

FIG. 13 is an enlarged front elevation view, in cross-section, of an alternative embodiment of an expanding guidewire apparatus in use in accordance with methods of the present invention.

FIG. 14 is a fragmentary side elevation view, in cross-section, of an alternative embodiment of FIG. 4, in the unexpanded state.

FIG. 15 is a fragmentary side elevation view of the alternative embodiment of FIG. 14, in the expanded state.

FIG. 16 is an enlarged, fragmentary top perspective view of the distal portion of the guidewire apparatus of FIG. 14.

FIG. 17 is a fragmentary side elevation view, in cross-section, of an alternative embodiment of the expandable guidewire in accordance with the present invention, and deployed in a medical device.

FIG. 18 illustrates an alternative geometry for the expandable portion of the guidewire in accordance with the present invention.

FIGS. 19A-19C illustrates an alternative embodiment guidewire apparatus in accordance with the present invention.

FIG. 20 is a fragmentary side elevation view, in cross-section, of the alternative embodiment of FIGS. 19B, and deployed in a medical device.

### DETAILED DESCRIPTION

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various Figures.

Referring now to FIGS. 2-5, an expandable guidewire apparatus 20 in accordance with the present invention is shown having an elongated main section 21, an expandable section 22, and a flexible tip section 23. The elongated main section 21 generally forms a substantial portion of the entire length of the guidewire apparatus, and defines an actuating lumen 25 extending substantially the length of the main section to a distal portion thereof. The expandable section 22 is coupled to the distal portion of the main section 21, and operably communicates with the actuating lumen 25 to facilitate selective actuation of the expandable section 22 between an unexpanded state (FIG. 2, 4) and an expanded state (FIGS. 3, 5). In the expanded state, a transverse cross-sectional dimension of the expandable section is greater than that in the unexpanded state. The guidewire apparatus further includes a flexible tip section 23 disposed distal to the expandable section 22, wherein the tip section is substantially more flexible than the elongated main section 21.

Accordingly, a guidewire apparatus 20 is provided that incorporates an expandable distal feature capable of selective radial expansion about its longitudinal axis. In the expanded state, the transverse cross-sectional dimension relative to that of the unexpanded stated can be controlled which enables several novel methods of use and applications. By way of example, as will be described in greater detail below, the expandable guidewire apparatus 20 is configured to join or lock together with an interior anchor wall 27 of a medical device 26, such as a catheter device, to facilitate torsion transmission from the guidewire apparatus 20 to the working element. As shown in FIGS. 5-7, the expandable section 22 may be caused to expand toward the expanded state until gripping contact with the interior anchor wall 27 of the catheter device 26 commences, frictionally joining the two independent components together as a unit. As is well known, torque can be more easily transmitted axially along a stiffer guidewire material to its distal portion than axially along a more flexible shaft material that typically comprises a catheter device. Accordingly, in this configuration, torque can be transmitted directly to the working element 28 (e.g., a dilation balloon) of the catheter device 26 to facilitate rotational alignment and vascular navigation thereof, via the coupled guidewire apparatus, rather than through the relatively flexible shaft of the catheter device.

As mentioned, and in further accordance with the present invention, the expandable section 22 may be frictionally joined with the interior anchor wall 27. Accordingly, in this configuration, axial loads can be transmitted directly to the medical device 26 through the expandable guidewire apparatus 20 to facilitate tracking through a vessel via the coupled guidewire apparatus, rather than through the relatively flexible shaft of the catheter device.

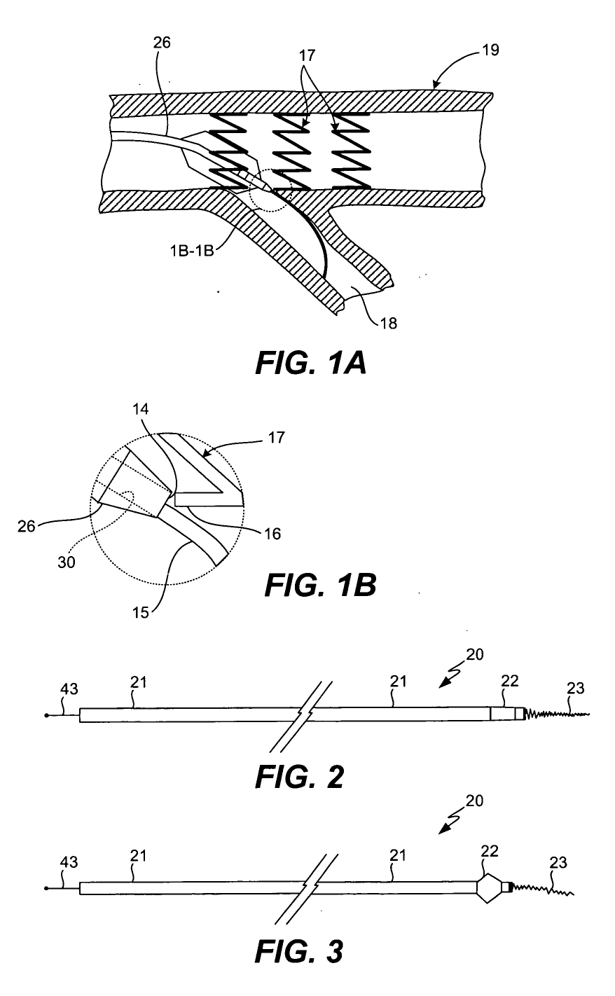

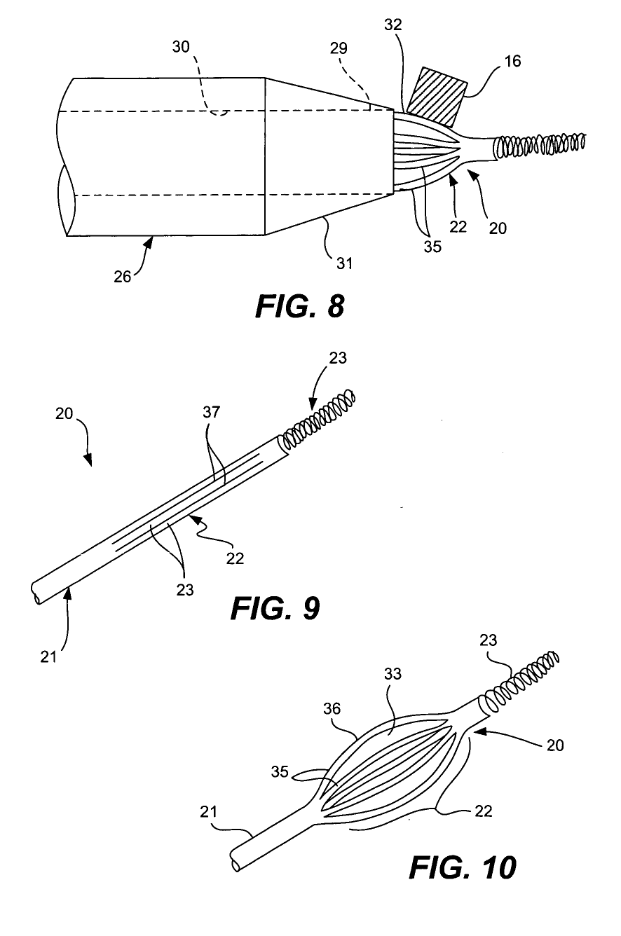

Another useful application of the guidewire expandable section 22 is to facilitate the pass through of the catheter tip through a deployed endoprosthesis device, such as the struts 16 of a deployed stent. As mentioned above, and as shown in FIG. 1, the disparity between the diametric widths of the guidewire and that of an opening 29 the guidewire lumen 30 at the tip of the catheter device 26 can sometimes cause the clearance phenomenon know as “fish-mouthing”. Consequently, the catheter tip may entangle the stent strut during passage through the strut cell and into a vessel side branch. In accordance with one specific application of the present invention, the expandable section 22 of the guidewire apparatus 20 can be deployed to the expanded state proximate to the guidewire lumen opening 29 (FIG. 8). In this configuration, the expandable section 22 fills the gap between the diametric width of the guidewire lumen 30 and the base diametric width of the guidewire, and forms a smoother radial transition longitudinally there along from the unexpanded distal tip section 23 of the guidewire apparatus 20 to the distal tip 31 of the catheter device 26. In essence, distal portion of the expandable section 22, in the expanded state, forms a smooth transition ramp 32 that enables relative sliding movement of the stent strut 16 past the catheter tip 31.

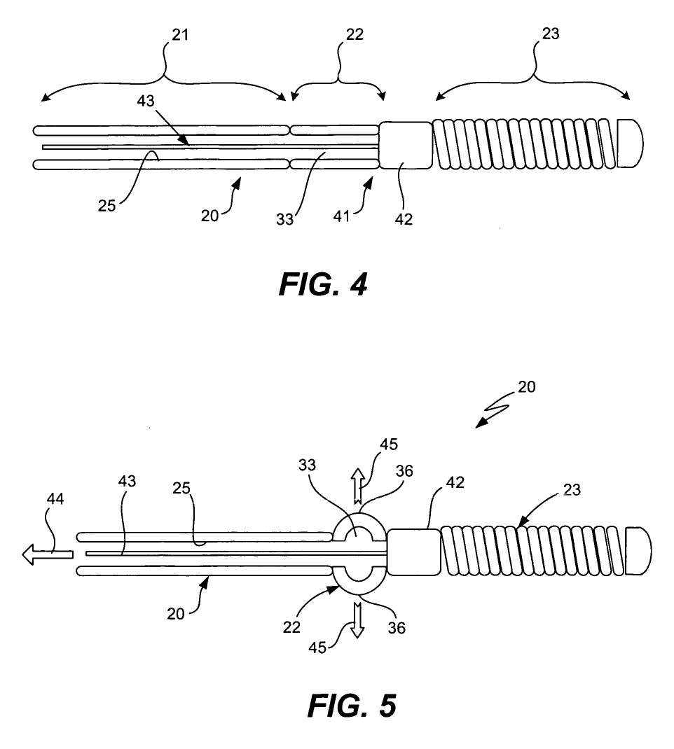

Referring back to FIGS. 2, 4, the expandable guidewire apparatus 20 is shown in a non-expanded state, wherein the expandable section 22 has a diameter or a transverse cross-sectional dimension substantially equivalent to that of the main section 21. The main section 21 of the expandable guidewire apparatus 20, briefly, may be provided by any conventional guidewire technology capable of supporting the actuating lumen 25 therethrough from a proximal end to a distal end thereof. Hence, the main section 21 is generally constructed of an elongated tubular member composed of a relatively rigid material, such as stainless steel, NITINOL, or a composite material such as a polymer that may or may not include reinforcing members.

As mentioned, the proximal end of the expandable section 22 is preferably mounted or affixed directly to, and is substantially co-axially aligned with, the distal end of main section 21. In one specific embodiment, the two sections are integrally formed with one another. In another embodiment, an intervening component may be disposed therebetween as well. Referring now to FIGS. 4 and 5, there is shown a cross-sectional view of the expandable section 22 in accordance with the present invention. The expandable section 22 comprises a proximal portion and a distal portion, and defines an interior lumen 33 in actuating communication with the main section actuating lumen 25 at the interface therebetween. Preferably, the diameter or transverse cross-sectional dimension of the interior lumen 33 at the proximal portion is sized similar to that of the actuating lumen 25 of the main section 21.

In one specific configuration, the expandable section 22 includes a plurality of expansion elements 35 disposed radially about a longitudinal axis of the expandable section. As best illustrated in FIGS. 9 and 10, each expansion element 35 resiliently bows radially outward such that the collective elements expand substantially transversely with respect to the longitudinal axis from the unexpanded state and the expanded state. Accordingly, as each expansion element 35 is urged toward the expanded state, a respective apex portion 36 of each element may be applied to contact a selected surface to secure the guidewire apparatus. For example, as illustrated in FIG. 7, the apex portions 36 of the expansion elements 35 securely contact and frictionally engaged against the interior anchor wall 27 of the catheter device 26 for secured mounting thereto.

FIG. 9 best illustrates that the expansion elements 35 may be defined by a plurality of substantially parallel elongated slits 37, each of which is disposed radially about, and extends substantially parallel to, the longitudinal axis of the expandable section 22. These slits 37 may be cut or formed directly into the elongate tube member utilizing known manufacturing processes such as laser cutting, water jet cutting, etching or similar processes. After being formed within the surface of the tube member, the expandable section may be heat treated to heat set thereby forming a section of the elongate tube member that can expand from one diameter to another without undergoing plastic deformation.

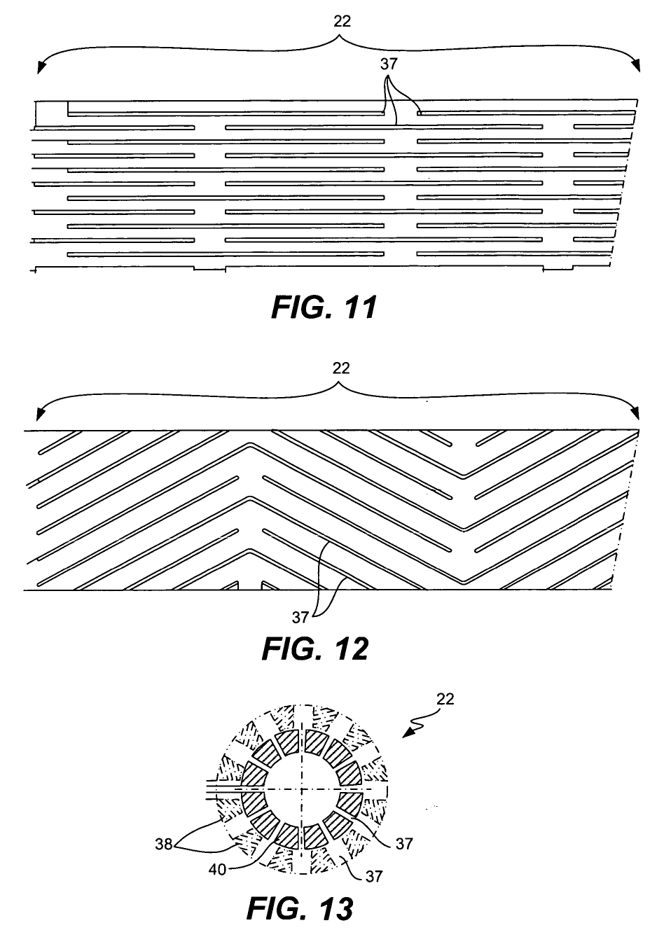

FIGS. 11 and 12 best illustrate alternative expansion patterns formed in the elongated tubular member that comprises the expandable section 22. FIG. 11, for instance, represents a staggered pattern generally comprising a plurality of substantially parallel extending slits 37, similar to the pattern of FIG. 9. Each adjacent slit 37 circumferentially extending about the periphery of the expansion extension, however, is longitudinally offset from one another in a repeating pattern. Such a configuration is especially suitable for maximizing expansion profile, and exhibits physical expansion properties such as higher actuation forces and maximized expansion profiles.

The expansion pattern shown in FIG. 12, by comparison, illustrates a plurality of nested V-shaped slits 37 in a herringbone pattern that again extend generally in a direction longitudinal to the expandable section 22. Also similarly, each adjacent V-shaped slit 37 is longitudinally offset from one another in a repeating pattern. Such a configuration is especially suitable for gradual expansion, and exhibits physical expansion properties such as low actuation forces and gradual expansion to maximum profile.

The expansion patterns shown in FIGS. 9, 11 and 12 are cut into the tubular member utilizing known processes such as laser cutting, wherein the tubular member is preferably NITINOL. After cutting, the tubular member can be grit blasted and electropolished as is known in the industry to smooth the edges of the cut section. The expandable section 22 may also be heat treated to remove residual stresses or to heat-set the expandable section in a desired shape.

Furthermore, the expandable section 22 may be constructed in a manner in which the expandable section is bi-stable, wherein a small force applied linearly to the expandable section will cause the elastic deformation as described herein, and when the force is removed the section will resiliently return to the substantially low profile, as shown in the unexpanded state of FIGS. 2, 4 and 9. Hence, essentially any elastometric material may be applied to the expandable section, including a polymer or a rubber material.

As mentioned, while the expandable section 22 in accordance with the present invention, is only illustrated as being a separate element in communication with the main section 21 of the guidewire, it is contemplated that the expandable section 22 may be integrally formed with the distal portion of the main section. In one configuration, by way of example, the main section 21 and the expandable section 22 may be formed from a NITINOL tube.

Alternatively, the expandable section 22 may be composed of a composite material having an outer elongated tubular member 38 and an inner reinforcement member 40 (FIG. 13). For example, one outer elongated tubular member 38 of the expandable section 22 may be constructed of an outer silicone material and be reinforced by an inner reinforcement tubular member 40. The inner reinforcement member, for instance, may be constructed of a metallic material or a composite material. Moreover, each tubular member 38, 40 composing the expandable section, may be patterned similarly or comprising a combination of expansion patterns, such as those of FIGS. 9, 11 and 12. Such combinations may be selected to utilize their specific physical expansion characteristics.

In still another alternative configuration, the expandable section 22 may be provided by an inflatable bladder device or the like, inflatable between the unexpanded state and the expanded state. In this embodiment, the inflatable bladder device defines an interior chamber that is in fluid communication with the actuating lumen 25 of the main section. Accordingly, the actuating lumen 25 functions as an inflationary lumen to control the diametric inflationary properties of the expandable section. As an inflationary material, the resilient bladder device may be constructed from a polymer material and a rubber material.

Referring to FIGS. 4-7, an actuating mechanism 41 cooperates with the expandable section for actuation thereof from the unexpanded state (FIG. 4, 6) to the expanded state (FIGS. 5, 7). This mechanism 41 includes a reciprocating actuator member 42 is disposed at the distal end portion of the expandable section 22. By applying a force to the actuator member 42 in a proximal direction, the actuator imparts an axial compressive force against the expandable section 22. The expansion elements 35 are thus caused to resiliently expand or buckle radially outward in response to the applied force. As shown, the collective effect increases the expandable section diameter in the expanded state.

The actuator member 42 is substantially cylindrical-shaped and is disposed in axial alignment with the expandable section 22. It is further contemplated that the proximal end of the actuator member 42 is configured to have a diameter substantially similar to that of the expandable section 22, thereby providing a smooth continuous surface for a medical device or the like to be slideably received on.

The actuating mechanism 41 includes a pull-wire device 43 having a distal portion anchored to the proximal end of the actuator member. A substantial length of the pull-wire device 43 is disposed in the actuating lumen 25 of the main section 21, having a distal portion extending through the interior lumen 33 of the expandable section 22 where the distal end thereof is joined, coupled and/or affixed to the actuator member. Accordingly, by applying an axial tension force to the pull wire device 43 in a proximal direction relative to the guidewire main section 21 (i.e., drawing the pull-wire proximally in the direction of arrow 44 in FIG. 5), the requisite axial compressive force is transmitted, via actuator member 42, to the distal portion of the expandable section 22. The expandable section is then caused to expand radially outward in the directions of arrows 45.

Upon release of the pull wire device 43, in the expanded state, the axial force imparted by the actuator member 42 is likewise released from the distal end of the expandable section. Consequently, the resiliency of the elastic expansion elements 35 urge the guidewire apparatus back toward the unexpanded state. In essence, the expandable section 22 returns to its original substantially low profile shape when the pull wire is released.

A removable handle (not shown) may be utilized in combination with the present invention. In this configuration, by way of example, the removable handle would be configured to receive the proximal portion of the guidewire apparatus main section. The actuating mechanism could further include a trigger device or the like that cooperates with a proximal portion of the pull wire device 43 to actuate the actuator member 42. The handle should be designed in a manner that is easily removable from the proximal end of the main section 21 of the guidewire apparatus 20 so that medical devices can be disposed over the guidewire itself. Moreover, the actuating mechanism can include a lock mechanism (not show) that can be engaged to lock the expandable section 22 in the expanded state, via cooperating with the trigger device and/or the pull-wire device.

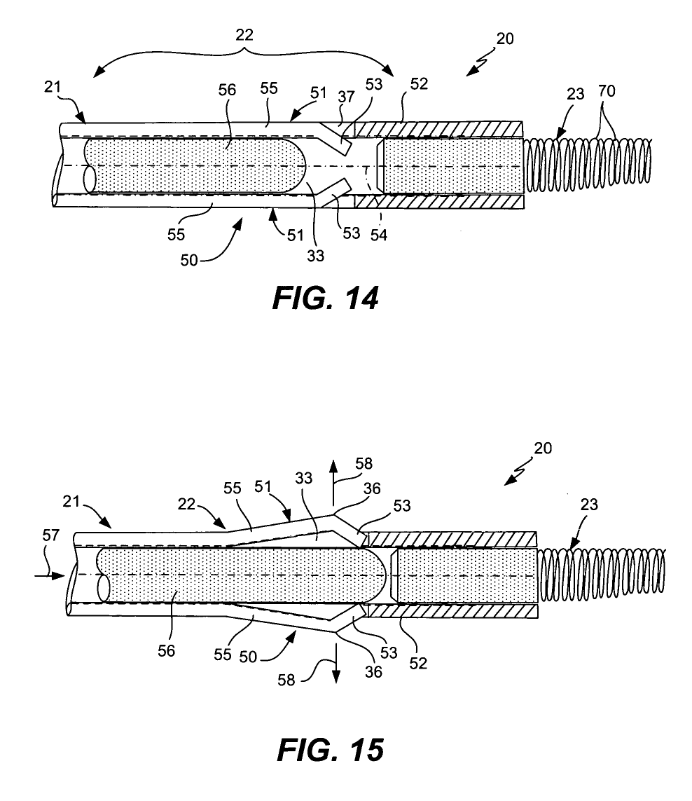

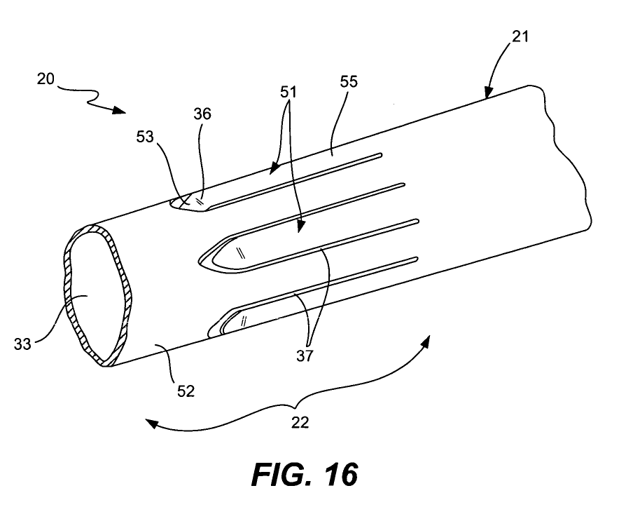

In yet another specific embodiment, referring now to FIGS. 14-16, an alternative expandable section 22 is disclosed that is operable by a push-wire actuator mechanism 50, as opposed to the pull-wire actuating mechanism 41 of the embodiments of FIGS. 4-7. In this configuration, a plurality of radially spaced expansion fingers 51 is formed in an elongate tubular member 52 of the guidewire expandable section 22. Similar to the previously disclosed embodiments, as best shown in FIG. 16, slits 37 may be cut or formed directly into the elongate tube member utilizing known manufacturing processes such as laser cutting, water jet cutting, etching or similar processes. Again, after being formed within the surface of the tube member, the expandable section may be heat-treated.

In accordance with the present invention, the distal tip portions 53 of the respective expansion fingers 51 are bent radially inward toward the longitudinal axis 54 of the expandable section 22. An apex portion 36 is thus formed between the distal tip portion 53 and a cantilever portion 55 that is cantilever mounted at respective proximal ends to the tube member 52. This arrangement provides spring-like radial expansion of the collective fingers 51 from the unexpanded state (FIG. 14) to the expanded state (FIG. 15) without undergoing plastic deformation.

By bending the distal tip portions 53 of the respective expansion fingers 51 into the interior lumen 33 of the expandable section 22, they are positioned for sliding contact with a push-wire 56 of the actuating mechanism 50. As shown in FIGS. 14 and 15, the push-wire 56 is sized and dimensioned for sliding axial movement distally in the direction of arrow 57 to engage the distal tip portions 53 of the expansion fingers 51.

To facilitate sliding contact with these distal tip portions 53, the distal end of the push-wire 56 is smoothly rounded. Once contact is commenced as the push-wire is urged distally, the distal tip portions 53 are urged radially outward in the directions of arrows 58 to the expanded state. Accordingly, the apex portions 36 are radially displaced outward by a sufficient distance for secured contact against the anchor wall 27 of the catheter device 26, for example.

By adjusting the angle of the inward bend of each distal tip portion 53, as well as its length, the radial displacement of each apex portion 36 from the longitudinal axis 54, in the expanded stated, can be adjusted. As shown in FIGS. 14 and 15, the angle is bend is acute, preferably ranging from about 15° to about 90°. Preferably, a radial expansion diameter of the apex portions 36 is selected in the range of about 0.015 inches to about 0.030 inches, although other diametric expansions can be selected as well.

To actuate the actuating mechanism 50, a removable handle (not shown) can be disposed at a proximal portion of the main section 21. Further, the actuating mechanism can incorporate a trigger device and locking mechanism at the removable handle, similar to the previously described embodiments.

While only one set of expansion fingers 51 is shown and described in detail, it will be appreciated that two or more sets of expansion fingers can be employed. In this configuration, each set can be axially spaced from one another, providing increased or altered locking characteristics.

Referring now to any embodiment of the expandable section 22, as shown in more detail in FIGS. 4-6 and 14-15, the deflectable or flexible tip section 23 of the guidewire apparatus 20 will now be described in detail. In one embodiment, the flexible tip section includes a proximal end mounted, coupled and/or joined to a distal end of the actuator member 42. Again, it will be appreciated that an intermediate component may be disposed therebetween as well. This tip section 23 is capable of being pre-bent to facilitate manipulation or navigation through a desired path. For example, after a first stent has been expanded against a target lesion in a bifurcated vessel, the balloon catheter device 26 may be withdrawn by an amount positioning the catheter tip proximate to the region of the bifurcation. The main guidewire may be withdrawn into the guidewire lumen 30, and the second guidewire apparatus 20, in accordance with the present invention, may be advanced therethrough. Once the second guidewire apparatus advances or is pushed out of the guidewire lumen 30, it will automatically take the shape of the pre-bent configuration. To facilitate positioning and orientation of the expandable section and/or distal tip section 23, radiopaque markers (not shown) may be disposed on any portion of the expandable guidewire apparatus 20.

By way of example, the flexible tip sections 23 may be constructed from a coiled wire such as a platinum coiled wire. The tip section 23 may further include a core member (not shown) around which coils 70 are disposed that provides additional support. A NITINOL tube material or spring, for example, can achieve such a pre-shaped or bent configuration. In another example, the flexible tip section may comprise a stainless steel spring or other polymeric or metallic pre-shaped configurations, which will force the catheter, tip to bend. Since the second guidewire apparatus 20 can freely rotate about its longitudinal axis within the inner guidewire lumen 30 of the medical device, the bent tip section 23 can easily be rotated and orientated to enter the vessel side branch through the strut cell. After a precise rotational and axial positioning of the flexible tip section 23, the guidewire apparatus 20 can be advanced towards and into the vessel side-branch.

In one specific use of the expandable guidewire apparatus, in accordance with the present invention, the expandable section 22 can be employed to improve side branch accessibility for the medical device. As mentioned above, and as shown in FIG. 1, the disparity between the diametric widths of the guidewire and that of the guidewire lumen opening 29 at the tip of the catheter device 26 can sometimes cause the clearance phenomenon know as “fish-mouthing”. Given the angularity of the stent strut 16, sometimes the catheter tip 31 can become entangled with the stent strut at this gap or clearance during passage through the strut cell.

In accordance with this specific application of the present invention, the expandable section 22 of the guidewire apparatus 20 is oriented and deployed to the expanded state proximate to the guidewire lumen opening 29 (FIGS. 8, 17 and 18). In this configuration, the expandable section 22 fills the gap between the diametric width of the guidewire lumen 30 and the base diametric width of the guidewire. By properly orienting and positioning the expandable section 22 in the opening 29 of the guidewire lumen 30, the inwardly tapered distal portion of the expandable section forms a smooth transition ramp or bridge. This smoothly sloped transition extends from the unexpanded distal tip section 23 (FIG. 8) of the guidewire apparatus 20 to the distal tip 31 of the catheter device 26, to significantly reduce any potential flaring, and enable relative sliding movement of the stent strut 16 past the catheter tip 31.

Once expandable section 22 is aligned, expanded and seated, the catheter device 26 and the guidewire apparatus 20 are temporarily locked together, and may be advanced together as a unit. This causes relative sliding axial movement of the stent strut 16 up the ramp 32 so that the catheter tip can pass into the side branch of a vessel or artery. Moreover, the expandable section can be configured to have a geometry that is complementary to the catheter tip and to form the smoothest transition between the catheter and the expanded portion of the guidewire.

FIG. 18 illustrates an alternative embodiment expandable section 22 that is shaped in a manner that is particularly suitable to function as a transition ramp 32 when moved to the expanded state. In particular, the expandable section 22 forms a conical shape that extends over the distal end edges that define the opening into the guidewire lumen of the catheter 26. Hence, an even smoother transition to the catheter tip is provided.

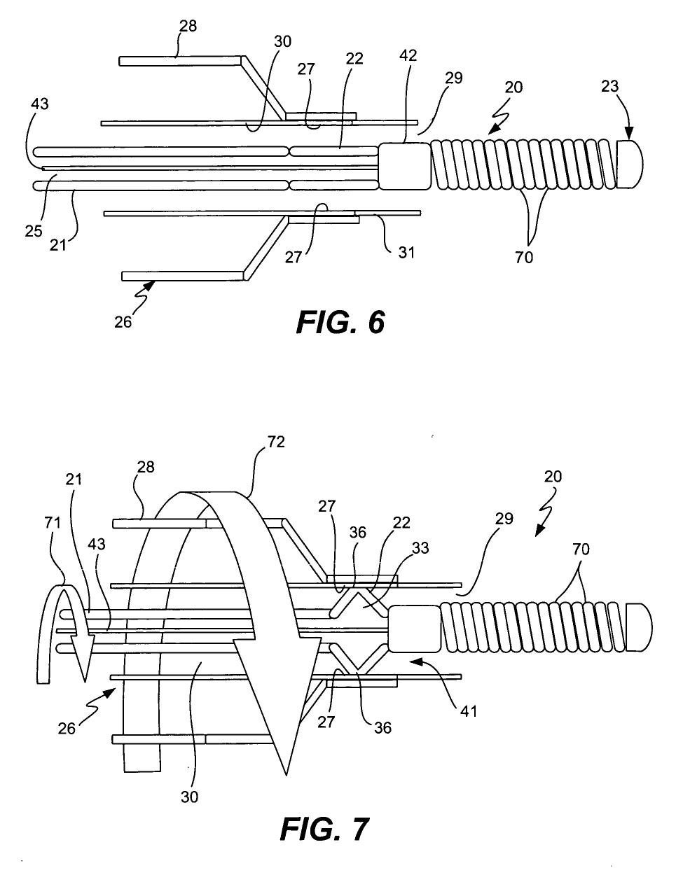

Referring back to FIGS. 6 and 7, another specific use of the present invention is disclosed. As previously mentioned, the expandable section 22 of the guidewire apparatus 20 may be deployed in a manner where, in the expanded state, the expandable section 22 cooperatively mates with the distal portion of the medical device 26, at or near the working element 28, to enable torque transmission from the guidewire to the medical device distal portion.

Due to the multiple bends in the anatomy of a coronary artery, the transmission of torque over the length the flexible shaft of catheter device 26 to its working element 28 (e.g., a dilation balloon) is very difficult, if not nearly impossible. Accordingly, it is often difficult to properly align and rotate the catheter device so that it can be advanced into a vessel side branch, for example. Additionally, it is difficult to properly align and rotate the catheter device so that the opening of a stent disposed thereon is properly positioned relative to a vessel branch ostium. In contrast, the shaft of a guidewire is significantly more stiff and rigid. By temporarily locking the expandable section 22 of the guidewire apparatus to the catheter working element 28 (FIG. 7), torque can be transmitted more easily down the length of the guidewire main section 21 to the catheter working element.

For this particular application, it may thus be advantageous to incorporate a main section 21 of the guidewire apparatus that is comprised of a relatively stiff material like a hypotube or a polymeric tube together with a stiffening wire. This combination can thus transmit considerably more torque.

During initial use, the main section 21, the expandable section 22 and the flexible tip section 23 of the guidewire apparatus 20 are advanced through the guidewire lumen 30 of the medical device 26 (or the medical device 26 is advanced over the guidewire apparatus). Due to the dimensional configurations between the components of the guidewire apparatus 20 and the diameter of the guidewire lumen 30, both the working element 28 of the catheter device 26 and the guidewire apparatus 20 can all freely rotate relative one another.

Once the flexible tip section 23 is advanced distally from the guidewire lumen opening 29 of the catheter tip, the pre-shaped flexible tip section of the guidewire apparatus may be positioned and rotatably aligned relative to the catheter tip 31 and with its targeted vessel (e.g., a side branch vessel). Applying radiopaque markings, the guidewire apparatus 20 can be axially advanced or withdrawn with precision, relative to the catheter guidewire lumen 30. When the expandable section 22 is positioned proximate to a targeted anchor wall 27, it is readied for deployment. Briefly, the interior anchor wall 27 that defines a portion of the guidewire lumen is preferably disposed inside of or proximate to the working element 28 and/or the catheter tip 31. While all the FIGURES illustrates placement of the anchor wall near the distal guidewire lumen opening, it will be appreciated, that the anchor wall could be disposed at a proximal portion of the working element 28. Moreover, it will be understood that the anchor wall 27 may essentially constitute any portion of the interior wall section that is selected for the expandable section 22 to engage against. In one specific embodiment, however, the interior anchor wall 27 may incorporate any additional reinforcing walls capable of increased resistance to radial deformation by the expansion section, when in the expanded state. For example, a preferred material would be selected that is harder and/or stronger than that composing the catheter tip portion. Such suitable materials include PEEK, polyimide, and stainless steel coiled wire.

Referring to FIG. 7, the expandable section 22 may be radially moved toward the expanded state, via actuating mechanism 41, until gripping contact commences between the apex portions 36 of each respective expansion element 35 and the interior anchor wall 27 of the catheter device 26. Further selective expansion to the expanded state causes frictional joining the two independent components together as a unit, substantially preventing relative rotational and axial displacement therebetween. In essence, the distal section of the expandable guidewire apparatus 20 is temporarily locked to the distal section of the medical device 26. At this point, a torque may be applied to the main section 21 of the guidewire apparatus 20 wherein the torque travels down its length to the expandable section. From here, the temporary locking of the guidewire apparatus to the medical device, via the expandable section 22 in the expanded state, enables torque transmission to the working element 28 of the medical device 26, as indicated in FIG. 7 by rotational arrows 71 and 72. Accordingly, in this configuration, torque can be transmitted directly to a working element 28 of the catheter device 26 to facilitate rotational alignment and vascular navigation thereof, via the coupled guidewire apparatus, rather than through the shaft of its own catheter device.

As mentioned, and in further accordance with this embodiment of the present invention, the expandable section 22 may be frictionally joined with the interior anchor wall 27. Accordingly, in this configuration, axial loads can be transmitted directly to the medical device 26 through the expandable guidewire apparatus 20 to facilitate tracking through a vessel via the coupled guidewire apparatus, rather than through the relatively flexible shaft of the catheter device.

It is contemplated that the guidewire and the medical device combination may be used to access either the side branch or main branch of a bifurcated vessel. In this way, the working element 28 of the catheter device 26 may be deployed thereby dilating the vessels.

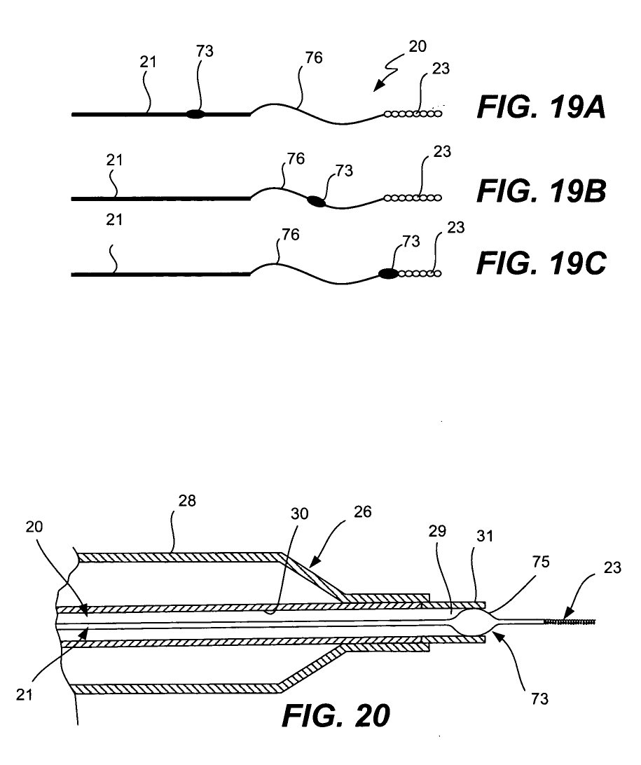

Referring now to FIGS. 19-20, another alternative embodiment guidewire apparatus 20 is disclosed in accordance with the present invention. Similar to the application of the expandable section 22 to form a smooth transition from the guidewire tip to the catheter tip, as best shown in FIG. 20, a non-expandable olive-shaped feature 73 may be provided along the guidewire apparatus that essentially performs the same function. In this embodiment, the elongated guidewire apparatus 20 (FIGS. 19A-19C) includes a similar, relatively stiff main section 21, an intermediate section 76 that is slightly more flexible than that of the main section 21, and a flexible tip section 23 similar to that of the previously described embodiments.

FIG. 20 illustrates that the feature 73 includes a diameter larger than that of the base wire of the main section 21 and the flexible tip section 23. The feature 73 is also ‘olive’ shaped, at least on its distal end side. Preferably, the feature is tapered inwardly on both sides thus allowing a gradual increase/decrease of the wire profile.

The feature 73 is formed on the outer surface of the guidewire apparatus 20 and is shaped to fit within and substantially fill the guidewire lumen opening 29 of the catheter device 26. Similarly, a distal portion of the feature 73 provides a smooth transition ramp portion 75 between the guidewire tip and the catheter tip. Unlike the expandable sections 22 described above, this olive-shaped feature 73 is non-expandable and is disposed on the outer surface of the guidewire. Similar to that described above, this feature 73 is applied and configured to substantially fill the gap of the guidewire lumen opening 29.

In accordance with the present invention, this guidewire apparatus 20 can be used in conjunction with a standard PTCA/SDS catheter device 26, as mentioned above, but also can be used to improve the ability of the catheter device to negotiate and pass through difficult anatomy or past implanted devices. As shown in FIG. 20, the balloon catheter/SDS device 26 can be advanced along the guidewire apparatus 20 until the catheter tip reaches the ‘olive’ feature 73. Due to the diameter of the ‘olive’ feature being larger than that of the base wire and the diameter of the guidewire lumen 30, the catheter device 26 cannot be advanced further.

Similar to the above-mentioned application for the expandable section 22, the catheter device becomes docked on the wire and the two devices can be advanced together as a single system. This has the advantage of improving the pushability of the system. Due to its tapered design, the ‘olive’ feature 73 can dock with the catheter tip 31 providing a close fit. This results in a catheter assembly featuring a temporary hard, which is highly advantageous for crossing through difficult anatomy or for passing the system by an already implanted device, such as through the struts of a stent, e.g. into the side branch vessel at a bifurcated anatomy.

Referring now to FIGS. 19A-19C, the ‘olive’ feature 73 may be positioned at different distal locations along the guidewire apparatus 20. For example, the feature can be located in either the stiffer main section 21 (FIG. 19A) or the flexible tip section 23 (FIG. 19C) of the guidewire. Finally, the feature 73 can be disposed in the intermediate section 76 as well (FIG. 19B).

By placing the ‘olive’ feature at the distal flexible tip section 23 location, the ability to cross through stent struts can be optimized. If the feature is placed too far distal on the flexible tip section 23, however, the wire will simply buckle or kink and cannot be pushed past the obstruction. The ‘olive’ feature 73 must be positioned at a location sufficiently proximal to the distal end of the flexible tip section 23 that allow enough wire to be positioned distally past the obstruction (e.g. through the stent struts and into the side branch at a bifurcation). Hence, the location of the ‘olive’ feature 73 should preferably be positioned at least about 3.0 cm proximally from the distal end of the guide wire. This configuration stabilizes the wire and allows the increased push of the wire and catheter combination to be channeled or guided in the desired direction.

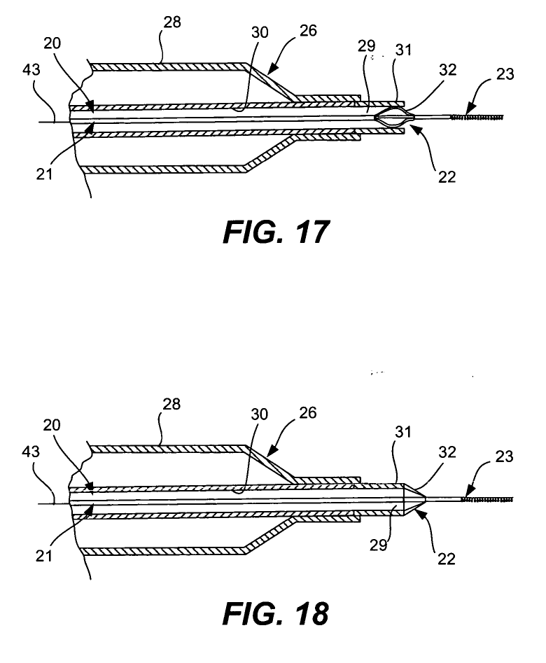

Again similar to the embodiments of FIGS. 9 and 17, the ‘olive’ feature 73 prevents the fish-mouthing from occurring as the ‘olive’ is seated snugly inside the catheter tip and does not allow flaring to occur. This is possible as the ‘olive’ fills the clearance between the wire and the catheter tip. The feature 73 provides a hard tip and a smooth transition ramp portion 75 between the profile of the base wire and the catheter tip upon which the catheter tip can therefore easily pass by the obstruction.

The ‘olive’ feature 73 is preferably constructed from a solid section comprised of a metal or hard polymer, and can be incorporated into all standard guidewires including pressure wires. In particular, the feature 73 may be constructed of a biocompatible metallic material such as platinum, silver, stainless steel, NITINOL, gold, tantalum or similar materials. Alternatively, the feature 73 may be formed of a biocompatible non-metallic material such as silicon, PVC, polyamide, cyanoacrylate or similar materials. In a preferred embodiment the feature 73 is affixed to the outer surface of the catheter, though it is contemplated that the feature may be rotationally disposed about the shaft of the guidewire, wherein a stop (not shown) may be disposed about the shaft of the guidewire to prevent the feature from traveling distally from a desired location.

While the preferred embodiment of the invention has been illustrated and described, it will be appreciated that various changes can be made therein without departing from the scope of the invention. It is therefore intended that the scope of the invention be determined from the following claims and equivalents thereof.

What is claimed is:

1. An expandable guidewire apparatus configured to temporarily mate with a medical device at a distal section thereof, said distal section including a working element and an interior wall defining a guidewire lumen extending through the working element and terminating at an opening thereof, said guidewire apparatus comprising:
an elongated main section having an actuating lumen extending substantially therethrough to a distal portion thereof, and
an expandable section coupled to the distal portion of the main section, and operably communicating with the actuating lumen to facilitate actuation of the expandable section between an unexpanded state and an expanded state, sized and dimensioned to temporarily mate against the interior wall of the guidewire lumen.

2. The guidewire apparatus according to claim 1, wherein
the expandable section includes an inflatable bladder device inflatable between the unexpanded state and the expanded state, said inflatable bladder device defining an interior chamber in fluid communication with the actuating lumen of the main section.

3. The guidewire apparatus according to claim 2, wherein
said bladder device is constructed from at least one of a polymer material and a rubber material.

4. The guidewire apparatus according to claim 1, wherein
said expandable section includes a plurality of expansion elements disposed radially about a longitudinal axis of the expandable section, and configured to expand substantially transversely with respect to said longitudinal axis from the unexpanded state and the expanded state.

5. The guidewire apparatus according to claim 4, wherein
said expandable section includes a plurality of generally longitudinally extending slits, defining said plurality of expansion elements.

6. The guidewire apparatus according to claim 5, wherein
the generally longitudinally extending slits are disposed in a pattern selected from a group consisting of a parallel pattern, staggered pattern and a herringbone pattern.

7. The guidewire apparatus according to claim 4, further including:
an actuator member disposed between expandable section and the flexible tip section; and
a pull wire device disposed in the actuating lumen of the main section, and having a distal end joined to said actuator member.

8. The guidewire apparatus according to claim 5, wherein
the expandable section is integrally formed with the main section.

9. The guidewire apparatus according to claim 4, wherein
said expandable section includes a tube member defining an interior lumen substantially co-axially aligned with the main section actuating lumen, and each said expansion element includes an expansion finger cantilever mounted to the tube member for movement between the unexpanded state and the expanded state.

10. The guidewire apparatus according to claim 9, wherein
each said expansion finger includes a cantilever portion having a proximal end cantilever mounted to the tube member, and a distal tip portion angled inwardly into the interior lumen at an acute angle, and
further including a push-wire member slideably disposed in the actuating lumen, and including a distal tip portion sized for sliding receipt in the interior lumen of the expandable section such that the push-wire distal tip portion is out of contact with the distal tip portions of the corresponding expansion finger, in the unexpanded state, and in contact with the distal tip portions to urge the corresponding expansion finger radially outward, in the expanded state.

11. The guidewire apparatus according to claim 1, wherein
said main section is selected from a group consisting of a NITINOL tube and a stainless steel tube.

12. The guidewire apparatus according to claim 1, further including:
a flexible tip section disposed distal to said expandable section, wherein the flexible tip section is substantially more flexible than the main section.

13. The guidewire apparatus according to claim 12, wherein
the flexible tip section includes a coiled portion.

14. A catheter system comprising:
a catheter device including an elongated tube member, and a distal section that includes a working element associated with a distal portion of the tube member, and a catheter tip portion associated with a distal end of the working element, said catheter device further defining a guidewire lumen extending through said elongated tube member and terminating at a distal end of the catheter tip portion, said distal section including an interior anchor wall defining a portion of the guidewire lumen; and
an expandable guidewire device disposed within said guidewire lumen of the catheter device, and configured for substantially axial displacement therein, said guidewire device including:
an elongated main section having an actuating lumen extending substantially therethrough to a distal portion thereof; and
an expandable section coupled to the distal portion of the elongated guidewire section, and operably communicating with the actuating lumen to facilitate selective actuation of the expandable section between an unexpanded state and an expanded state, having a transverse cross-sectional dimension greater than that in the unexpanded state and sufficient to contact and frictionally engage the interior anchor wall of the distal section by an amount such that selective torsional and/or axial forces applied to the elongated guidewire section are transmitted to the distal section of the catheter.

15. The catheter system according to claim 14, wherein
said interior anchor wall is composed of a material that is harder than the catheter tip portion to radially reinforce the catheter tip portion.

16. The catheter system according to claim 14, wherein
said expandable section includes a plurality of expansion elements disposed radially about a longitudinal axis of the expandable section, and configured to expand substantially transversely with respect to said longitudinal axis from the unexpanded state and the expanded state.

17. The catheter system according to claim 16, wherein
said expandable section includes a plurality of longitudinally extending slits, defining said plurality of expansion elements.

18. The catheter system according to claim 16, further including:
an actuator member disposed distal to the expandable section;
a pull wire device disposed in the actuating lumen of the main section, and having a distal end joined to said actuator member.

19. The catheter system according to claim 16, wherein
said expandable section includes a tube member defining an interior lumen substantially co-axially aligned with the main section actuating lumen, and each said expansion element includes an expansion finger cantilever mounted to the tube member for movement between the unexpanded state and the expanded state.

20. The catheter system according to claim 19, wherein
each said expansion finger includes a cantilever portion having a proximal end cantilever mounted to the tube member, and a distal tip portion angled inwardly into the interior lumen at an acute angle, and
further including a push-wire member slideably disposed in the actuating lumen, and including a distal tip portion sized for sliding receipt in the interior lumen of the expandable section such that the push-wire distal tip portion is out of interference contact with the distal tip portions of the corresponding expansion finger, in the unexpanded state, and in contact with the distal tip portions to urge the corresponding expansion finger radially outward, in the expanded state.

21. The catheter system according to claim 14, further including:
a flexible tip section disposed distal to said expandable section.

22. A method for temporarily and selectively mating a guidewire apparatus to a distal section of a medical device disposed in a vessel to transfer an a force from the guidewire apparatus to the medical device, said distal section including a working element and an interior wall defining a guidewire lumen extending through the working element and terminating at an opening thereof, said method comprising:
positioning an expandable guidewire apparatus, having an elongated main section and an expandable section associated with the main section, in the guidewire lumen such that the expandable section is oriented proximate to the medical device distal section;
actuating the expandable section of the guidewire apparatus from an unexpanded state, sized and dimensioned for substantially interference free axial and rotational passage through the guidewire lumen, to an expanded state, sized and dimensioned to temporarily engage the guidewire lumen interior wall at the distal section of the medical device; and
applying a force to the main section of the guidewire apparatus that is transmitted to the working element of the medical device via the temporarily mated expandable section of the guidewire apparatus, in the expanded state.

23. The method as recited in claim 22, wherein
said applying a force includes applying a torsion force to the main section.

24. The method as recited in claim 22, wherein
said applying a force includes applying an axial force to the main section.

25. The method as recited in claim 22, wherein
said actuating the expandable section includes radially expanding a plurality of expansion elements disposed radially about a longitudinal axis of the expandable section, and configured to elastically deform radially outward with respect to said longitudinal axis from the unexpanded state and the expanded state.

26. The method as recited in claim 25, wherein
said radially expanding the plurality of expansion elements includes pulling a pull-wire proximally, having a distal end joined to an actuator member disposed distally to the expandable section, such that the actuator member substantially axially compresses said expandable section toward the expanded state.

27. The method as recited in claim 25, wherein
said expandable section includes a tube member defining an interior lumen, and each said expansion element includes an expansion finger cantilever mounted to the tube member for movement between the unexpanded state and the expanded state, and
said radially expanding the plurality of expansion elements includes pushing a push-wire, longitudinally disposed in an actuating lumen of the guidewire main section, in a direction distally such that said push wire slideably contacts respective distal tip portions of the expansion fingers angled inwardly into the interior lumen to urge the corresponding expansion finger radially outward, in the expanded state.

28. The method as recited in claim 22, further including:
releasing the expandable section to release the engagement with the guidewire lumen interior wall such that the medical device distal section and the expandable section are not rotationally coupled, and the expandable section elastically returns to substantially the unexpanded state.

29. A method for reducing potential flaring between a guidewire and a distal section of a medical device inserted over the guidewire during a medical procedure, said distal section of the medical device including a working element and defining a guidewire lumen extending through the working element and terminating at a distal opening thereof, said method comprising:
orienting the guidewire, having an elongated main section and an expandable section associated with a distal portion of the main section, in the guidewire lumen such that the expandable section is positioned proximate to the distal opening into the guidewire lumen of the medical device;
actuating the expandable section of the guidewire apparatus from a base diameter unexpanded state, sized and dimensioned for substantially interference free axial and rotational passage through the guidewire lumen, to an expanded state, sized and dimensioned to substantially fill the guidewire lumen opening of the distal section to form a relatively smooth transition ramp, tapering radially outward, from the base diameter of the guidewire to at least substantially transverse cross-sectional dimension of the guidewire lumen opening at the distal section of the medical device.

30. The method as recited in claim 29, further including:
applying an axial force to the medical device wherein the transition ramp formed by the expand section facilitates passage through a resistant location.
